(19) United States
(12) Patent Application Publication
(10) Pub. No.: US 2023/0023418 A1
(43) Pub. Date: Jan. 26, 2023

(54) PREDICTING USER MOUTH LEAK USING COMPUTER MODELING

(71) Applicant: ResMed Pty Ltd, Bella Vista (AU)

(72) Inventors: Liam HOLLEY, Bella Vista (AU); Ji Hae JANG, Bella Vista (AU); Jamie WEHBEH, Bella Vista (AU)

(21) Appl. No.: 17/872,930

(22) Filed: Jul. 25, 2022

(30) Foreign Application Priority Data
Jul. 26, 2021 (AU) 2021902284

(51) Int. Cl. G16H 10/60

(52) U.S. Cl. CPC G16H 10/60

(57) ABSTRACT

Techniques for improved model-based predictions are provided. Patient data for a patient associated with a positive airway pressure (PAP) therapy is accessed, and a set of features is extracted from the patient data. A first predicted mouth leak measure is generated by processing the set of features using a leak model, and in response to determining that the first predicted mouth leak measure satisfies defined criteria, provisioning of a first PAP apparatus for the patient is facilitated.

### CROSS-REFERENCE TO RELATED APPLICATIONS

This application claims benefit of and priority to Australian Provisional Patent Application No. 2021902284, filed Jul. 26, 2021, which is herein incorporated by reference in its entirety.

### INTRODUCTION

Embodiments of the present disclosure relate to predicting events using computer modeling. More specifically, embodiments of the present disclosure relate to using machine learning and other models to predict patient issues with ongoing therapy.

Positive airway pressure (PAP) therapies (such as continuous PAP (CPAP)) apply pressure to the upper airways. There are many parameters that influence the likelihood of a patient succeeding with therapy, such as therapy mode, interface comfort, therapy pressure, interface adjustments, and the like. When therapy is delivered via the nose only (as opposed to the nose and mouth), it is possible for the patient to breath or leak air through their mouth. Air leaks from the mouth can cause drying of the nasal and oral passages, which can be uncomfortable, painful, and harmful to the tissues and functions of the airways. As a result, mouth leaks can result in reduced adherence to PAP therapy, and can often be a major cause for patients to quit therapy altogether.

However, for many patients, mouth leak is not a problem, so generic solutions (such as suggesting or using a full-face PAP mask that covers the mouth) fail to adequately solve these issues. In conventional systems, the only way to identify and solve mouth leak is to begin the therapy and closely monitor the patient to determine whether mouth leak is occurring. This is time-consuming, costly, and can lead to substantial discomfort for the patient.

Improved systems and techniques to provide accurate and reliable therapy predictions are needed.

### SUMMARY

According to one embodiment presented in this disclosure, a method is provided. The method includes: accessing patient data for a patient associated with a positive airway pressure (PAP) therapy; determining a mouth leak measure of the patient during the PAP therapy; extracting a set of features from the patient data; generating a predicted mouth leak measure by processing the set of features using a leak model; and refining the leak model based on a difference between the mouth leak measure and the predicted mouth leak measure.

According to one embodiment presented in this disclosure, a method is provided. The method includes: accessing patient data for a patient associated with a positive airway pressure (PAP) therapy; extracting a set of features from the patient data; generating a first predicted mouth leak measure by processing the set of features using a leak model; and in response to determining that the first predicted mouth leak measure satisfies defined criteria, facilitating provisioning of a first PAP apparatus for the patient.

Other aspects provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example workflow to use leak models to predict therapy issues and facilitate improved treatments.

FIG. 2 depicts an example workflow to generate leak models to predict therapy issues.

FIG. 3 is a flow diagram depicting an example method for generating and updating leak models based on patient data.

FIG. 4 is a flow diagram depicting an example method for using leak models to identify optimal therapy devices.

FIG. 5 is a flow diagram depicting an example method for extracting features from patient facial data.

FIG. 6 is a flow diagram depicting an example method for extracting features from patient informational data.

FIG. 7 is a flow diagram depicting an example method for generating predicted leak measures for therapy devices using leak models.

FIG. 8 is a flow diagram depicting an example method for training leak models.

FIG. 9 is a flow diagram depicting an example method for facilitating provisioning of therapy devices using leak models.

FIG. 10 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved predictions relating to therapy outcomes.

In some embodiments, leak models are disclosed that enable accurate and reliable prediction of mouth leak in PAP therapy patients. For example, a machine learning model (e.g., a neural network, a convolutional neural network, a regression model, and the like) may be trained to generate predicted leak measures indicating predictions such as a probability that the patient will experience mouth leak, an amount of predicted mouth leak, and the like. In some aspects, alternative solutions or treatment strategies may be used in order to reduce or prevent such mouth leak, even before therapy has begun.

In some embodiments, the treatment strategies for eliminating or reducing the occurrence of mouth leaks, may include actions such as suggesting or providing the patient with an interface that applies therapy pressure to both the nose and the mouth, providing a chin strap or other device to reduce the mouth opening, mouth sealing tape (to seal the mouth during therapy), a mouth guard, or mandibular advancement device, and the like. As discussed in more detail below, there is substantial advantage to being able to predict whether a patient is likely to benefit from a therapy system that reduces the risk of mouth leak before the patient has even started on therapy for the first time. This advantage can be particularly relevant in situations where the patient is set up remotely (for example, if their therapy equipment is mailed to them). In such situations, if the equipment is found to be less than ideal (e.g., due to mouth leak), then side-effects of therapy (e.g. dry and irritated airways) will be more likely to negatively impact the patient experience, and the motivation for therapy can be greatly reduced. Further, the patient may need to wait a considerable time to have the situation rectified (or they may lose motivation in the waiting period, and as a result be less likely to achieve therapy adherence in the future).

In some embodiments of the present disclosure, techniques and systems are provided to predict the probability that a particular patient will have issues (e.g., mouth leak) with particular therapy systems or components thereof, and/or the probability that particular alternative systems or components thereof would reduce such issues or not cause such issues at all. For example, the techniques described herein may be used to predict the likelihood that a CPAP patient will experience problematic mouth leak if they were set-up with a nasal-only interface. Predicting therapy issues on PAP therapy systems before therapy onset can be significantly advantageous, as it may allow better selection of therapy apparatus or therapy settings for the patient and improved personalization of therapy based on patient data.

In some aspects of the present disclosure, a variety of patient characteristics or attributes (such as height, weight, facial anatomy, and the like) are used to predict mouth leak. In some aspects, other extrinsic data can additionally or alternatively be evaluated to predict mouth leak. As an example, in some embodiments, data relating to the environment where the patient sleeps (e.g., the temperature, humidity, air pollution levels, types of air pollution present, and the like), or other data that may be predictive of nasal congestion (such as the time of year or season, location or region where the patient lives, and the like) may be used as input to predict mouth leak. For example, air pollution may cause rhinitis, which may increase nasal flow resistance and in turn increase the probability that the patient will experience mouth leak. Generally, aspects of the present disclosure may use a wide variety of intrinsic and/or extrinsic data to predict mouth leak or other therapy issues.

In embodiments, one or more leak models can be used to predict leak measures, such as the probability that an individual will have issues with mouth leak and/or the predicted volume of such mouth leak. In some embodiments, a leak model can be used to predict mouth leak if the patient uses only a nasal mask. That is, the leak model may receive input such as patient attributes, and generate a mask-agnostic prediction as to whether mouth leak is a potential concern for the patient. In some embodiments, a leak model may predict mouth leak measures based at least in part on proposed therapy data, such as a proposed mask model and/or type, particular therapy pressures, particular patient orientations, and the like. That is, the model may use such therapy data as input in order to generate therapy-specific predictions (e.g., mask-specific predictions, pressure-specific predictions, and the like). In some embodiments the system may use such a model to evaluate multiple alternative therapy options, allowing it to identify or generate therapy options that are less likely to result in mouth leak issues, and/or or warn against a number options that are likely to result in mouth leak issues. For example, the system may propose a range of mask models, therapy pressures, and other accessories that may be suitable for the patient.

### Example Workflow to Use Leak Models to Predict Therapy Issues and Facilitate Improved Treatments

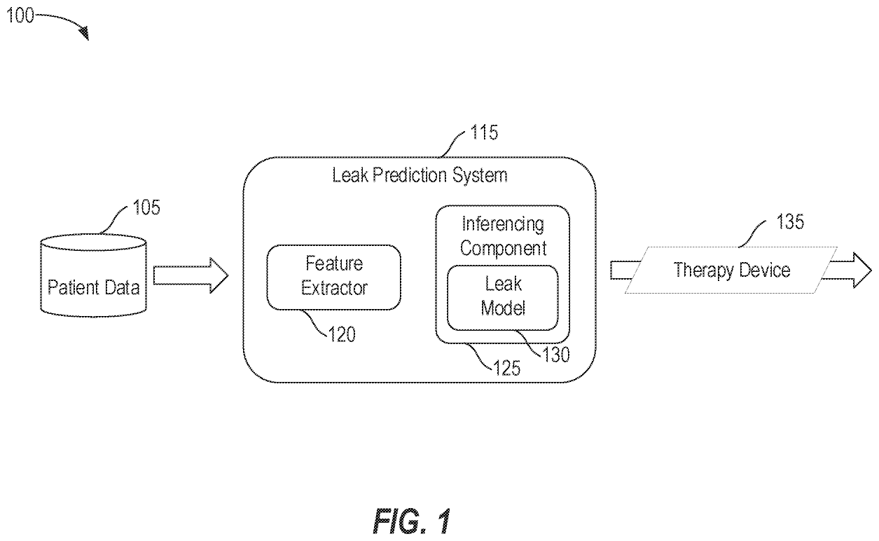

FIG. 1 depicts an example workflow 100 to use leak models to predict therapy issues and facilitate improved treatments.

In the illustrated workflow 100, a set of patient data 105 is used by a leak prediction system 115 to generate a suggested therapy device 135. Although depicted as a discrete system for conceptual clarity, in some aspects, the leak prediction system 115 may be implemented as one or more components of a broader system, in the cloud, and the like. The operations of the leak prediction system 115 may generally be implemented using hardware, software, or a combination of hardware and software, and may be combined or distributed across any number and variety of components.

In the illustrated aspect, the patient data 105 is provided to or accessed by the leak prediction system 115. That is, the patient data 105 resides external to the leak prediction system 115 in the illustrated example. In embodiments, however, the leak prediction system 115 may access the patient data 105 from any suitable location, including from internal storage. In at least one aspect, some or all the patient data 105 is provided (e.g., by a user) of the leak prediction system 115 in order to output the therapy device 135. In some aspects, some or all of the patient data 105 may be automatically retrieved or accessed by the leak prediction system 115. For example, when a user requests a leak prediction be generated for a given patient, the leak prediction system 115 may automatically identify and retrieve the relevant patient data 105.

Although depicted as a single data store for conceptual clarity, in embodiments, the patient data 105 may reside in a variety of data stores and locations. For example, some of the patient data 105 may reside in a user profile maintained by one entity or data store, some in a medical history stored by one or more care providers, and the like.

As discussed below in more detail, the patient data 105 generally includes or indicates a variety of data, attributes, and/or characteristics of one or more patients. As used herein, the term “patient” can include an individual who is actively engaged in therapy (e.g., PAP therapy), as well as prospective patients (e.g., individuals who are candidates for or may begin therapy, but who are not yet engaged in the therapy). For example, a first patient may be a user who has a CPAP device and has been using the therapy for some time, while a second patient may be a user who is considering beginning CPAP therapy.

In some aspects, the information included in the patient data 105 corresponds to data or attributes that have been determined to have predictive power with respect to mouth leak. For example, a subject matter expert may indicate specific attributes that contribute to mouth leak. In at least some embodiments, the predictive attributes are determined using one or more feature selection techniques. For example, the leak prediction system 115 may automatically evaluate a variety of data to find attributes that are highly-correlated with mouth leak (e.g., above a defined threshold) in order to determine which features to use in predicting mouth leak for new patients.

In some aspects, the features may be selected using a hybrid of manual and automated techniques. For example, one or more clinical subject matter experts may identify suspected features to include in the model during a research phase (or for testing using a simplified model), such as if the feature is suspected to be highly causal (e.g., high nasal impedance or small nostril airpath cross-sectional area) with a potential advantage of lower computation or data storage costs. In one such embodiment, for features that are not clearly predictive, the system may use automated feature extraction techniques and more complex algorithms.

Generally, the patient data 105 can include a wide variety of information, depending on the particular implementation. For example, in some embodiments of the present disclosure, the patient data 105 may include data such as demographics of the patient, the age of the patient, physical characteristics of the patient (e.g., height, weight, body-mass index (BMI), and the like), facial geometry information (e.g., one or more images of the patient's face and/or neck, one or more two-dimensional and/or three-dimensional scans of the patient's face and/or neck, measurements of various facial features (e.g., nose width and height, neck circumference, etc.), and the like.

As additional non-limiting examples, the patient data 105 may include data such as vocal sound recordings of the patient (which may be evaluated to infer various nasal and/or throat geometries), medical history of the patient, an acoustic impedance measurement (which may be used to infer nasal resistance to airflow), survey responses from the patient (e.g., indicating whether they have a history of experiencing mouth breathing and/or dry mouth).

In some embodiments, the patient data 105 can include data indicative of resistance to air flow through the nasal passage of the patient. For example, the patient data 105 may include information related to the size and shape of the nose of the patient, the cross-sectional area of the nasal vestibule of the patient, the hydraulic radius (e.g., the ratio of the circumference to the area), and/or data that is indicative of inflammation of the nasal passage (e.g., a diagnosis of rhinitis). Because nasal resistance to air flow may increase the likelihood of oral breathing, such features may be included in the patient data 105 (if available).

In some aspects, the patient data 105 can additionally or alternatively include a variety of features extracted from responses to one or more questionnaires or surveys. For example, the patient may be asked to confirm, rate, or otherwise describe symptoms or experiences such as difficulty breathing through their nose, nasal congestion, rhinitis, dry mouth, drool, any anatomical or other condition that may impair the ability to maintain a mouth seal during sleep, or previous experience of mouth leak on CPAP therapy.

In some embodiments, the patient data 105 may include information indicative of the lung volume of the patient. For example, a large lung volume may be associated with large respiratory air flow rates, which may increase the likelihood of oral breathing (e.g., mouth leak).

In some embodiments of the present disclosure, the patient data 105 may include information indicative of the mechanics of the mandible and connected tissues of the patient. For example, in some aspects, the amount of movement of the mandible and/or the amount of force on the mandible may affect the likelihood of oral breathing, as discussed in more detail below.

In some embodiments, the patient data 105 may include information indicative of the mechanics of oral sealing of the patient. For example, the leak prediction system 115 may determine the anatomical parameters of the patient's mouth which may be predictive of the likelihood of mouth leak, such as the thickness and/or size of the patient's upper and/or lower lips, the length, depth, and/or angle of the mentolabial sulcus of the patient, the volume of the upper and/or lower pars marginalis muscles of the patient, and the like.

In some aspects, the patient data 105 can include a variety of features explicitly (e.g., explicitly indicating demographics of the patient, age of the patient, weight of the patient, and the like). In some aspects, the patient data 105 can additionally or alternatively include information that can be used to determine or estimate a variety of features. For example, the patient data 105 may include one or more images or scans of the patient's face or head, which may include some or all of the neck. Such images or scans may be processed to extract various features, such as nose size, as discussed in more detail below.

In at least one embodiment, some or all of the patient data 105 is collected from a potential or prospective PAP patient (e.g., as part of the screening or diagnostic process). As discussed above and in more detail below, such data may include, without limitation: diagnostic data, photographic data, voice sound recordings, breathing sound recordings, snore sound recordings, respiratory flow signals, lung volumes, answers to survey questions (e.g., demographic, physiological, genetic, anatomical, and/or psychological details), and/or details about disease symptoms provided by the patient. As additional non-limiting examples, the patient data 105 may include information such as health records, retail history, technology and device usage patterns may (e.g., obtained using wearable devices, fitness tracking devices or applications, heart monitoring devices, medication tracking systems, implantable medical devices, or other medical devices or consumer products). As additional examples, the patient data 105 may additionally or alternatively be collected using medical therapy or diagnostic devices, such as determining therapy parameters, disease parameters, therapy usage patterns, and/or physiological data.

In the illustrated workflow 100, the leak prediction system 115 includes a feature extractor 120 and a leak model 130. Though two discrete components are depicted for conceptual clarity, in some aspects, the operations of the depicted components (and others not depicted) may be combined or distributed across any number and variety of components and systems.

In the illustrated example, the feature extractor 120 can be used to process the patient data 105 to extract one or more features (e.g., attributes or characteristics) that are or may be predictive of mouth leak. In at least one embodiment, the feature extractor 120 can additionally provide other preprocessing, such as to segment input images or scans (e.g., to identify, extract, and/or crop to the region(s) of the input data that depict the nose, mouth, or other features of the user), which may reduce or eliminate confounding variables in the process. Similarly, in some aspects, the feature component 120 may perform preprocessing operations such as outlier detection, data smoothing, noise removal, and the like, depending on the particular input data type.

The extracted features can generally vary depending on the particular implementation. For example, from two-dimensional and/or three-dimensional neck and facial geometry in the patient data 105, the feature extractor 120 may extract data that can be used in analysis of the mechanics of the movement of the mandible and the sealing of the mouth. As one example, the feature extractor 120 may use the patient data 105 to predict the likely range of movement of the mandible of the patient, relative to the maxilla, in a patient who has musculature atonia associated with sleep. Such features can then be used to predict the likelihood that the mouth would remain sealed under the conditions of a given range of movement, therapy pressures, respiratory flow patterns, and the like, as discussed in more detail below. In some aspects, the feature extractor 120 may evaluate a geometric model of the patient to extract features such as the mentolabial sulcus angle, the radius of curvature surrounding the geniohyoid, and the like.

In some embodiments, as discussed above, the leak prediction system 115 can determine, infer, or predict various aspects of the anatomy and forces of the patient's mandible in order to perform mouth leak prediction. As discussed above, some or all of the factors involved in such analysis may be explicitly included in the patient data 105, while some or all of the relevant factors may be inferred or extracted by the feature extractor 120.

In one such embodiment, the feature extractor 120 may access or use a model of the muscular and joint forces applied to the mandible and use patient-specific data in the patient data 105 to extract relevant features. For example, based on one or more images or scans of the patient's face and/or neck, the feature extractor 120 may determine or infer data such as the spatial coordinates or locations of the fulcrum of the patient's mandible, and/or various connection points for muscles and/or organs of the patient to the mandible and/or other structures (e.g., to the skull), such as the posterior connection point of the temporalis muscle, the medial connection point of the temporalis muscle, the anterior connection point of the temporalis muscle, the connection point(s) of the deep masseter muscle, the superficial masseter muscle, the superior head of the lateral pterygoid muscle, the inferior head of the lateral pterygoid muscle, the medial point of the pterygoid muscle, and the like. In some aspects, in addition to or instead of extracting the connection points of such organic structures the feature extractor 120 can extract or infer attributes such as muscle tone of various muscles, the predicted gravitational acceleration on the mandible (e.g., based at least in part on sleeping position, such as whether the patient sleeps supine or upright), and the like.

In some embodiments, a to determine or infer the anatomy of the patient's face, the leak prediction system 115 may use a simplified structural-mechanical model of the facial skeleton such that the model can be fit to a parameterized model of the facial surface. In one example, the facial surface model may be fit to facial surface imagery and/or a three-dimensional face scan of the patient, and the skeletal structural-mechanical model may be fit to the facial surface model. For example, the structural model may include locations in space relative to three orthogonal planes, such as the sagittal, coronal, and transverse planes, that define the location of various features, such as, the centroids of the temporomandibular joints, the most anterior point of the mandible on the sagittal plane, the most anterior point on the maxilla on the sagittal plane, and the like. In some embodiments, the structural-mechanical skeletal model may include degrees of freedom of the mandible relative to the maxilla, or the reference planes, such as rotation of the mandible about the temporomandibular joints, or anterior-posterior movement of the mandible. As such, in an embodiment, it is possible to estimate alternative locations of features on the surface of the face based on modelled movement of the mandible. In some embodiments, alternative feature locations may be included as features in the system. In some embodiments, forces exerted by gravitational acceleration, and reaction forces on the structures, may additionally or alternatively be included in the structural-mechanical model, such that a range of static equilibrium positions for various parts of the structure may be estimated. In some aspects, facial feature locations may be calculated for potential static equilibrium positions and these locations may be used as features in a classifier.

Generally, the feature extractor 120 may perform such mandible anatomy evaluation to extract features, from the patient data 105, which may be useful in force analysis for the mandible of a sleeping patient. In some aspects, during such sleep, there may be partial or significant atonia in the musculature connected to the mandible. In some embodiments, therefore, the feature extractor 120 may identify and/or extract features of the anatomy that correspond to or affected by gravitational acceleration, rather than strictly forces that are produced by muscle activation.

Therefore, in some embodiments, based on two-dimensional and/or three-dimensional imagery and/or geometry of the patient's face and/or neck, the feature extractor 120 may extract features that may be predictive of the geometry of the mandible and maxilla, and the volume, or mass of organs or other tissue, such as muscle or fat deposits that are connected with the mandible. For example, in some embodiments, the feature extractor 120 estimates the coordinates of the location of the temporomandibular joint (e.g., the fulcrum of the mandible), as well as gravitational force vectors applied to the mandible by connecting tissues (e.g., based on their connection points). The feature extractor 120 (or other components of the leak prediction system 115) can then determine or infer reaction forces of the mandible needed to keep it in a stationary static equilibrium while the patient sleeps (e.g., using classical static mechanics methods or techniques).

In at least one embodiment, to enable prediction of the reaction forces of the mandible, the feature extractor 120 may estimate the mass of one or more organs, such as muscles and/or fat attached to the patients' mandible. In one such embodiment, the feature extractor 120 may fit patient data 105 to a model of such organ(s) in order to infer the organ mass with respect to the individual patient. For example, the feature extractor 120 may use partial information about the patient's anatomy, such as their neck and facial surface geometry, body mass index, neck circumference, and the like, in order to fit to a model of the organ(s) to the patient data. This fitted model can then be used to estimate mass of the organ(s). In one embodiment, the organ mass is estimated based on extrapolating the surface geometry enclosing a particular organ to estimate the size, shape, and/or composition of that organ. For example, the leak prediction system 115 may construct an estimate of the structure of the geniohyoid muscles from the one surface enclosed by skin tissue under the chin. From the estimated structure, the system can then estimate masses of the organs. For example, the leak prediction system may estimate the volume of muscle tissue in a particular organ, and multiply the estimated volume by an average facial muscle density (e.g., the average of similar patients having similar attributes or characteristics, such as similar height, weight, BMI, and the like) in order to determine or estimate the mass of that muscle. Using such predicted masses (as well as other data in some aspects, such as the mass and connection points of the connecting muscles or other tissue or organs, such as connecting points on a skeletal structural model) as well as the likely orientation of gravitational acceleration acting on the anatomy (e.g., based on reported or preferred sleeping position of the patient or commonly known sleeping positions), the feature extractor 120 can better predict the force on the patient's jaw, thereby enabling prediction of whether the jaw will open sufficiently to enable mouth leak.

In some embodiments, the feature extractor 120 (or another component, such as the inferencing component 125) may compare the determined or inferred reaction forces to a range of typical values expected to be produced by the corresponding muscles of a sleeping patient of a similar type (e.g., having a similar weight, height, or other characteristic to the patient).

In the illustrated example, the inferencing component 125 receives the relevant patient features (e.g., included in the patient data 105 and/or extracted by the feature extractor 120) to predict whether the patient will experience mouth leak on PAP therapy (e.g., using one or more leak models 130. In some embodiments, the leak model 130 corresponds to one or more trained machine learning models. For example, the leak model 130 may comprise one or more artificial neural networks, joint probability architectures, or Baysian classifiers, support vector machines, and the like. In some embodiments, the leak model 130 is formed as a combination or ensemble of various models (or any other methods or models used for of classification, regression, or prediction).

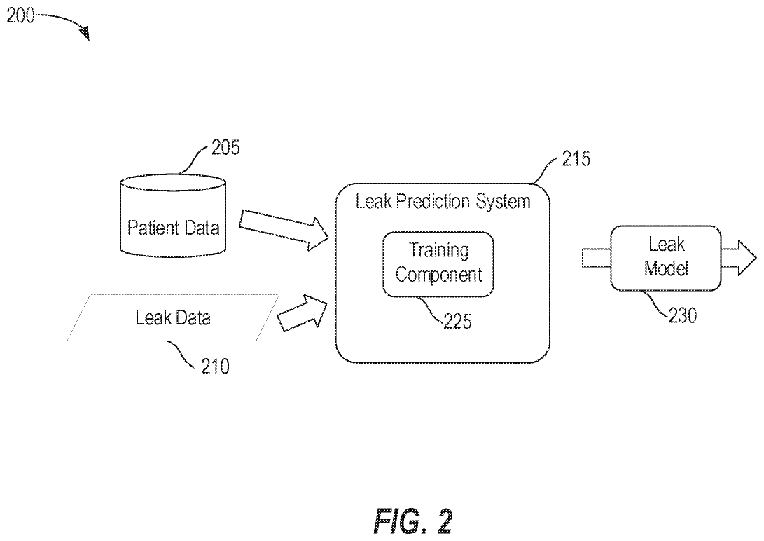

In some aspects, the leak model 130 can include one or more rules-based evaluations. For example, in at least one embodiment, as discussed above, the inferencing component 125 may compare the estimated muscle reaction forces needed to maintain static equilibrium of the mandible with the predicted force needed (e.g., determined based on features such as organ mass and connection points). In some embodiments, the reasonably expected muscle forces of the patient may be determined or inferred based on data such as the patient's muscle mass and/or using comparison to other similar patients with known muscle forces (e.g., by identifying other patients with similar physical attributes such as height, weight, and age, and determining the average or median muscle force available to hold the mandible of such patients closed). By comparing the predicted gravitational force and the expected muscle force, the inferencing component 125 can predict the probability or likelihood of the mandible moving during sleep, and thus determine the probability or risk of mouth leak during PAP therapy. Some examples relating to the generation or training of the leak model 130 are discussed in more detail below with reference to FIG. 2.

Generally, the specific operations of the leak model 130 may vary depending on the particular implementation. For example, in one embodiment, the leak model 130 is a convolutional neural network that receives image(s) of the patient's face and/or neck (or segments of images that correspond to these features), and generate an output leak measure based on these images. In some aspects, the leak model 130 may additionally or alternatively receive other data, such as patient demographics, physical characteristics of the patient (e.g., height, weight, facial anatomy and/or geometry, distance and/or angles of facial features, etc.), and the like, and generate a corresponding predicted leak measure. Similarly, in some aspects, the leak model 130 may receive or predict the mandibular forces (e.g., attributed to gravitational force on the jaw caused by attached organs) and/or mandible retention forces (e.g., attributed to muscles of the patient) in order to predict the leak measure.

In the illustrated example, using the leak model 130, the leak prediction system 115 can generate and output one or more indications of one or more therapy devices 135. That is, the leak prediction system 115 may indicate suggested alternative therapy devices for the patient, where the indicated device(s) may be less likely to result in mouth leak (or likely to result in reduced mouth leak). For example, in one such embodiment, the leak prediction system 115 may evaluate multiple alternative therapy devices using the leak model 130 in view of the patient data 105. By generating a predicted leak measure for each alternative (e.g., a predicted amount of leak and/or probability of leak), the leak prediction system 115 can identify the optimal therapy device(s) (e.g., those having the lowest predicted amount of leak and/or lowest probability of mouth leak).

In some embodiments, the leak prediction system 115 outputs the therapy devices 135 as suggestions to a user (e.g., to the patient and/or to a care provider). For example, via a graphical user interface (GUI), the leak prediction system 115 may indicate that one or more specific device configurations are likely to be optimal for the specific patient. For example, the suggested therapy device 135 data may indicate or suggest use of a full face mask (e.g., as opposed to a nasal-only mask), use of other assistance such as a chin strap, and the like.

In some aspects, the user (e.g., care provider) can then use the recommendations to provision a therapy device for the patient (e.g., by ordering one from a supplier). For example, the care provider may approve or accept the suggestion, and the leak prediction system 115 (or another system) may automatically order or request the suitable device for the patient. In at least one embodiment, the leak prediction system 115 (or another system) can automatically provision the therapy device without such input.

Although not included in the illustrated example, in some aspects, the leak prediction system 115 can additionally or alternatively output the generated leak measures. For example, for a set of alternative therapy devices 135, the leak prediction system 115 may indicate the respective predictions for each alternative. This may allow the user or other systems to select the optimal device or apparatus for specific patients. For example, the leak prediction system 115 (or another system) may select the therapy device 135 having the lowest probability of mouth leak, the therapy device 135 having the lowest predicted volume of mouth leak, or the therapy device 135 satisfying or balancing a combination of such factors.

In these ways, the leak prediction system 115 facilitates provisioning of improved therapy devices for specific patients, thereby improving the therapy results and outcomes. That is, patients can receive improved results, as compared to conventional approaches. This can improve therapy compliance, reduce adverse side effects, and generally improve patient outcomes. In embodiments, the leak prediction system 115 can additionally provide computational improvements in various ways. For example, in at least one aspect, the leak prediction system 115 can generate a predicted leak measure for a given index device (e.g., for the therapy device that is currently used by the patient, or the device that the patient intends to use for the therapy). If the predicted leak measure is within allowable criteria (e.g., with a probability of leak and/or volume of leak below one or more thresholds), the leak prediction system 115 may determine that the indicated mask is sufficient, and refrain from further processing. That is, the leak prediction system 115 may refrain from evaluating any alternative masks using the leak model 130, as the indicated or current mask is acceptable. This can substantially reduce computational expense.

In one such aspect, if the current or indicated mask does not satisfy the criteria (e.g., because the probability and/or predicted volume of mouth leak exceed one or more thresholds), the leak prediction system 115 may optionally or selectively process alternative devices and/or suggestions (such as suggesting a chin strap, suggesting the user sleep in a different position, and the like) using the leak model 130. By only selectively evaluating such alternatives, the leak prediction system 115 can reduce the computational resources needed to generate adequate and improved suggestions.

In some aspects, the leak prediction system 115 may additionally or alternatively predict or estimate mouth leak at various therapy pressure settings. For example, the leak prediction system 115 may use the leak model 130 to predict mouth leak based on the patient data 105 at a first PAP pressure, to predict mouth leak at a second PAP pressure, and so on. Based on these pressure-specific predictions, in one embodiment, the leak prediction system 115 may determine or identify if there is a threshold therapy pressure at which a threshold mouth leak flowrate is likely to be exceeded. The system may then alert a user, or automatically modify therapy settings or prescriptions. For example, a maximum therapy pressure may be automatically set to a value that limits the likelihood of an undesirable level of mouth leak flow.

### Example Workflow to Generate Leak Models to Predict Therapy Issues

FIG. 2 depicts an example workflow 200 to generate leak models to predict therapy issues.

In In the illustrated workflow 200, a set of patient data 205 and leak data 210 is used by a leak prediction system 215 to generate a leak model 230. Although depicted as a discrete system for conceptual clarity, in some aspects, the leak prediction system 215 may be implemented as one or more components of a broader system, in the cloud, and the like. The operations of the leak prediction system 215 may generally be implemented using hardware, software, or a combination of hardware and software, and may be combined or distributed across any number and variety of components.

In at least one aspect, the leak prediction system 215 corresponds to the leak prediction system 115 of FIG. 1. That is, a single leak prediction system may train or generate leak models, as well as use them during runtime to predict mouth leak measure for patients. In other embodiments, the leak prediction system 215 may differ from the leak prediction system 115. That is, a first system or device may train or generate the leak models, which may then be used by one or more other systems or devices to generate leak measure during runtime.

In the illustrated aspect, the patient data 205 is provided to or accessed by the leak prediction system 215. As discussed above, the patient data 205 may reside in any suitable location, including external to the leak prediction system 215 and/or in internal storage. Although depicted as a single data store for conceptual clarity, in embodiments, the patient data 205 may reside in a variety of data stores and locations. For example, some of the patient data 205 may reside in a user profile maintained by one entity or data store, some in a medical history stored by one or more care providers, and the like.

In some aspects, the patient data 205 may be alternatively referred to as training data, training exemplars, historical data, and the like. The patient data 205 generally comprises information relating to patients that are currently on PAP therapy and/or were previously on PAP therapy, where attributes of the therapy (e.g., the actual leak data 210 during the therapy) are known (or where subjective experience (e.g., patient-reported leak issues) may be known). In this way, the patient data 205 can be used to train or generate leak models.

As discussed below in more detail, the patient data 205 generally includes or indicates a variety of historical data, attributes, and/or characteristics of one or more patients. In at least one embodiment, the patient data 205 can include similar or the same data as the patient data 105 of FIG. 1. That is, the information included in the patient data 205 may correspond to data or attributes that have been determined to have predictive power with respect to mouth leak. For example, in some embodiments of the present disclosure, the patient data 205 may include data such as demographics of patients, the ages of the patients, physical characteristics of the patients (e.g., height, weight, body-mass index (BMI), and the like), facial geometry information (e.g., one or more images of the patients' face and/or neck, one or more two-dimensional and/or three-dimensional scans of the patients' face and/or neck, measurements of various facial features (e.g., nose width and height, neck circumference, etc.), and the like.

In the illustrated workflow 200, the leak prediction system 215 also receives leak data 210. In some aspects, the leak data 210 includes information relating to mouth leak experienced by one or more patients during PAP therapy. For example, for each patient reflected in the patient data 205, the leak data 210 may indicate the therapy device or apparatus they used (e.g., the type of PAP mask, such as a full-face mask, nose-only mask, with or without a chin strap, and the like), as well as whether they experienced mouth leak (and, in some embodiments, the amount of mouth leak they experienced). In this way, the patient data 205 and leak data 210 can collectively be used as training exemplars to train one or more leak models.

In the illustrated example, the leak prediction system 215 includes a training component 225. Although not depicted in the illustrated example, in some aspects, the leak prediction system 215 may additionally or alternatively include other components, such as a feature extractor (e.g., feature extractor 120 of FIG. 1), inferencing component (e.g., inferencing component 125 of FIG. 1), and the like. In embodiments, the training component 225 may be implemented using hardware, software, or a combination of hardware and software.

Generally, the training component 225 uses the input training data (e.g., the patient data 205 and leak data 210) to generate or train one or more leak models 230 (which may correspond to the leak model 130 of FIG. 1). In some embodiments, the leak model 230 can be trained on any set or combination of data, including any of the types of data or features extracted from the data mentioned previously. For example, samples from patients known to have experienced mouth leak or other therapy issues (as indicated in the leak data 210) and samples from patients known not to have experienced mouth leak or other therapy issues can be included in the training data. In this way, the training component 225 can train the leak model 230 built to predict a classification or regression value for new patients (including those yet to receive therapy, patients who are at an early stage their therapy experience, and/or to identify patients who may benefit from alternative methods of providing therapy, such as an alternative interface).

In at least one example, to train the leak model 230, the training component 225 processes a set of patient data 205 for a given historical patient using a given therapy device at a given time using the leak model in order to generate a predicted leak measure (e.g., a probability that the patient experienced mouth leak, a predicted amount of mouth leak the patient experienced, and the like). This prediction can then be compared against the known leak data 210 for the given patient at the given time/using the given device. Based on the difference between the predicted leak and actual leak, the training component 225 can refine or update the parameters of the leak model in order to generate improved predictions. For example, in the case of a neural network, the training component 225 may use backpropagation to update the parameters using gradient descent.

As discussed above, the output of the leak model 230 may collectively be referred to as leak measures, and can include one or more classifications (e.g., indicating whether the patient will experience or is likely to experience mouth leak) and/or one or more numerical values or scores (e.g., indicating a probability that the patient will experience mouth leak, indicating a predicted volume of the mouth leak, and the like).

In some embodiments, as discussed above, the output predictions are conditioned on therapy apparatuses or options. For example, the leak model 230 may consider, as input, whether the patient will use a full-face mask, a chin strap, a nasal-only mask, and the like. In other embodiments, the leak model 230 may predict the leak measures independently of such factors, and users can use the predictions to determine whether specific therapy options are a good fit. For example, patients classified by the leak model 230 as likely to experience mouth leak may be provided with a full face mask and/or a chin strap, and/or other equipment or coaching (such as providing a humidifier, increasing humidification settings of the PAP device, and the like) in order to prevent or alleviate the issue.

In the illustrated workflow 200, the trained leak model 230 can then be deployed to one or more systems for inferencing. For example, as discussed above with reference to FIG. 1, systems may use the leak model 230 to generate leak measures for current and/or prospective patients of PAP therapies, thereby enabling significantly improved outcomes and results.

In embodiments, the workflow 200 may be used in response to any number of criteria or events. For example, the workflow 200 may be manually triggered (e.g., by a user or administrator), or may be automatically triggered based on specified criteria. In one embodiment, the leak prediction system 215 (or another system) can initiate the workflow 200 upon determining that new training data is available, upon determining that the model accuracy has degraded (e.g., where patient feedback indicates that the suggestions were not helpful or did not prevent mouth leak), and the like.

### Example Method for Generating and Updating Leak Models Based on Patient Data

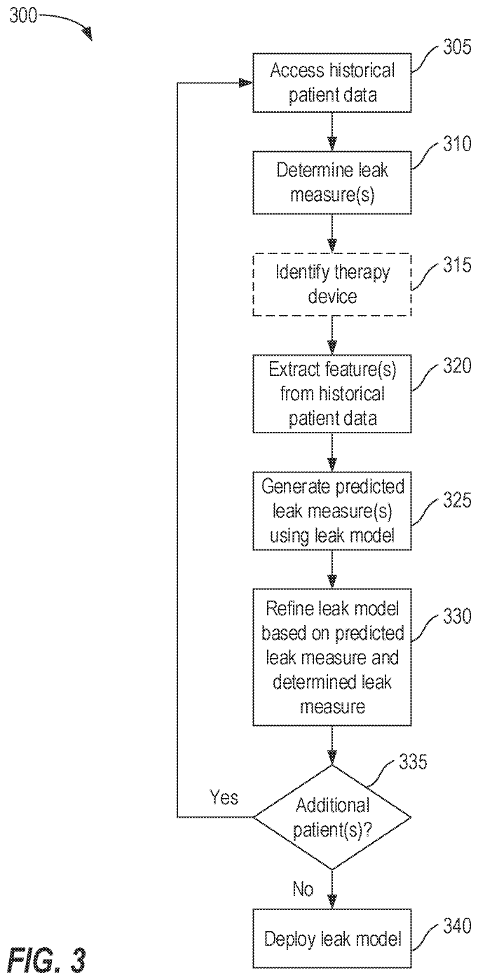

FIG. 3 is a flow diagram depicting an example method 300 for generating and updating leak models based on patient data. In some embodiments, the method 300 is performed by a leak prediction system, such as leak prediction system 215 of FIG. 2, and/or leak prediction system 115 of FIG. 1.

At block 305, the leak prediction system accesses historical patient data. As used herein, “accessing” data may generally refer to requesting it, receiving it, retrieving it, collecting it, or otherwise gaining access to it for processing. As discussed above, the historical patient data may generally correspond to data for one or more patients that are currently engaged in and/or previously engaged in one or more therapies, such as PAP therapy. That is, the historical patient data may include information for patients where therapy issues (such as mouth leak) are known and/or have been quantified. In at least one aspect, the historical patient data corresponds to the patient data 205 of FIG. 2.

In one aspect, accessing the patient data at block 305 corresponds to accessing data for a specific patient at a specific point in time, and/or a specific patient with respect to a specific reported therapy issue. For example, the leak prediction system may select patient data corresponding to a reported instance of mouth leak for a specific patient. Generally, the techniques used to select the specific patient data can vary, as all available historical data can be evaluated using the method 300.

At block 310, the leak prediction system determines one or more leak measure for the accessed historical data. For example, as discussed above, the leak prediction system may determine whether the patient that corresponds to the accessed historical patient data experienced mouth leak using the corresponding therapy device, an amount of the mouth leak, and the like. In at least one aspect, the leak measure corresponds to leak data 210 of FIG. 2.

At block 315, the leak prediction system optionally identifies the therapy device(s) used by the patient at the time. For example, as discussed above, the leak prediction system can determine whether the user used a nasal pillow mask, a nasal mask, a full-face mask, an oral mask, and the like. In some aspects, the leak prediction system can additionally or alternatively determine whether any further therapy devices or apparatus were used, such as a humidifier, a chin strap, and the like.

In the illustrated example, block 315 is indicated as optional to reflect that the training of the model may or may not depend on the particular mask used by the patient. That is, in some embodiments, the specific mask type and configuration may be used as input to the leak model to generate a predicted leak measure with respect to the specific patient using the specific apparatus(es). In other embodiments, the leak model may process patient data to generate a device-agnostic predicted leak measure that is not premised on the mask type used.

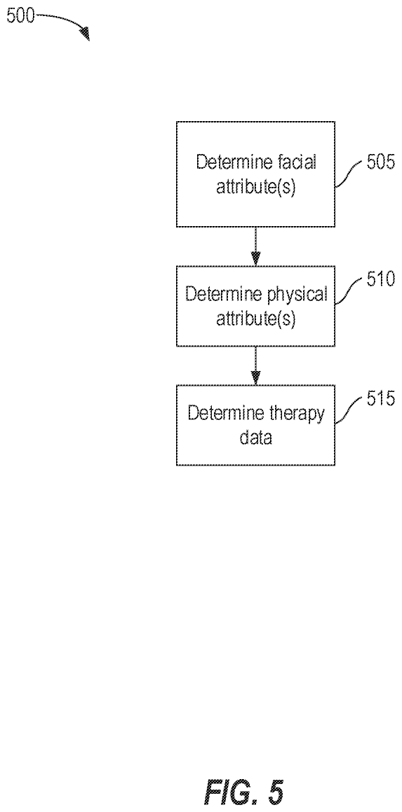

At block 320, the leak prediction system extracts one or more features from the historical data. For example, the leak prediction system may use a feature extractor (such as feature extractor 120 of FIG. 2) to perform preprocessing (such as segmentation of images based on the patient's nose) and/or other operations to extract the features. As discussed above, the extracted features generally correspond to characteristics or attributes that may be predictive of mouth leak in patients. For example, the extracted features may include data such as facial geometry or measurements, nose anatomy, anatomy of the patient's mandible and organs attached thereto, and the like. Additional details relating to feature extraction are discussed in more detail below with reference to FIGS. 5 and/or 6.

At block 325, the leak prediction system generates one or more predicted leak measures for the historical patient data by processing the accessed patient data using a leak model, as discussed above. For example, the leak model may comprise one or more machine learning models trained to predict mouth leak based on patient characteristics. As discussed above, the particular implementation of leak model may vary, and may include models such as convolutional neural networks (e.g., to generate leak predictions based on images of the patient's face and/or neck), artificial neural networks (e.g., to process categorical or numerical data, such as patient demographics and physical descriptors, in order to predict leak), rules-based systems (e.g., to compare predicted gravitational forces on the mandible and predicted muscular forces on the mandible), and the like.

In some embodiments, an ensemble of models is used. For example, one model may process image data to generate predicted leak measures while a second processes biographical data to predict mouth leak and a third processes facial anatomy or geometry to predict forces on the patient's mandible. By combining predictions from multiple models, the leak prediction system may be able to achieve improved prediction accuracy.

At block 330, the leak prediction system refines the leak model(s) based on the predicted leak measure(s) and the determined leak measure(s) (determined at block 310). For example, the leak prediction system may compute a loss based on the difference between the predicted and actual leak measures, and use backpropagation to refine the parameters of the model. Generally, the specific operations used to refine the model parameters may vary depending on the particular model architecture used.

At block 335, the leak prediction system determines whether there is at least one additional historical patient that has not yet been evaluated/used to refine the model. If so, the method 300 returns to block 305. If not, the method 300 continues to block 340. Although the illustrated example uses the presence of additional training data as the terminating criteria for the training process, in some embodiments, other termination criteria may be used. For example, the leak prediction system may determine whether a defined period of time or amount of computational resources have been used performing the training, whether the model accuracy meets defined minimum criteria, and the like.

At block 340, the leak prediction system deploys the trained leak model. As discussed above, deploying the model can generally include deploying it locally (e.g., where the leak prediction system uses it to process patient data) and/or deploying it to one or more other systems (e.g., to dedicated inferencing systems). The leak model can then be used to generate accurate leak measures for therapy patients, thereby significantly improving their results.

In embodiments, the method 300 may be used in response to any number of criteria or events. For example, the method 300 may be manually triggered (e.g., by a user or administrator), or may be automatically triggered based on specified criteria. In one embodiment, the leak prediction system (or another system) can initiate the method 300 upon determining that new training data is available, upon determining that the model accuracy has degraded (e.g., where patient feedback indicates that the suggestions were not helpful or did not prevent mouth leak), and the like.

### Example Method for Using Leak Models to Identify Optimal Therapy Devices

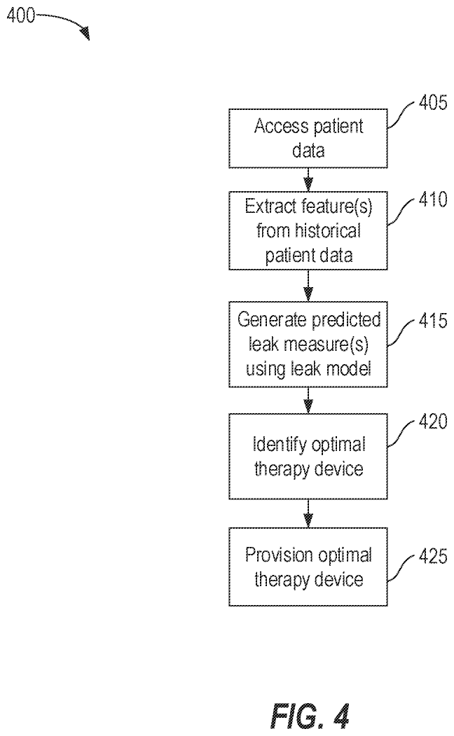

FIG. 4 is a flow diagram depicting an example method 400 for using leak models to identify optimal therapy devices. In some embodiments, the method 400 is performed by a leak prediction system, such as leak prediction system 115 of FIG. 1, and/or leak prediction system 215 of FIG. 2.

At block 405, the leak prediction system accesses patient data. As discussed above, the patient data may generally correspond to data for one or more patients that are currently engaged in and/or may begin to engage in one or more therapies, such as PAP therapy. That is, the patient data may include information for patients that are currently using PAP therapy, as well as prospective patients that are considering PAP therapy. In at least one aspect, the historical patient data corresponds to the patient data 105 of FIG. 1.

In one aspect, accessing the patient data at block 405 corresponds to accessing data for a specific patient at the current point in time. For example, the leak prediction system may select or receive patient data corresponding to a patient that is currently beginning therapy, is about to begin therapy, and the like.

In some embodiments, as discussed above, the leak prediction system can optionally identify the therapy device(s) used by the patient (in the case of current therapy patients). For example, as discussed above, the leak prediction system can determine whether the patient is using a nasal pillow mask, a nasal mask, a full-face mask, an oral mask, and the like. In some aspects, the leak prediction system can additionally or alternatively determine whether any further therapy devices or apparatus were used, such as a humidifier, a chin strap, and the like.

As discussed above, identifying the specific therapy being used may be optional, as the patient may or may not actually be engaged in the therapy. Father, in embodiments, the model may or may not depend on the particular mask used by the patient. That is, in some embodiments, the specific mask type and configuration may be used as input to the leak model to generate a predicted leak measure with respect to the specific patient using the specific apparatus(es). In other embodiments, the leak model may process patient data to generate a device-agnostic predicted leak measure that is not premised on the mask type used.

At block 410, the leak prediction system extracts one or more features from the patient data. For example, the leak prediction system may use a feature extractor (such as feature extractor 120 of FIG. 2) to perform preprocessing (such as segmentation of images based on the patient's nose) and/or other operations to extract the features. As discussed above, the extracted features generally correspond to characteristics or attributes that may be predictive of mouth leak in patients. For example, the extracted features may include data such as facial geometry or measurements, nose anatomy, anatomy of the patient's mandible and organs attached thereto, and the like. Additional details relating to feature extraction are discussed in more detail below with reference to FIGS. 5 and/or 6.

At block 415, the leak prediction system generates one or more predicted leak measures for the patient data by processing the accessed patient data using a leak model, as discussed above. For example, the leak model may comprise one or more machine learning models trained to predict mouth leak based on patient characteristics. As discussed above, the particular implementation of leak model may vary, and may include models such as convolutional neural networks (e.g., to generate leak predictions based on images of the patient's face and/or neck), artificial neural networks (e.g., to process categorical or numerical data, such as patient demographics and physical descriptors, in order to predict leak), rules-based systems (e.g., to compare predicted gravitational forces on the mandible and predicted muscular forces on the mandible), and the like.

In some embodiments, an ensemble of models is used. For example, one model may process image data to generate predicted leak measures while a second processes biographical data to predict mouth leak and a third processes facial anatomy or geometry to predict forces on the patient's mandible. By combining predictions from multiple models, the leak prediction system may be able to achieve improved prediction accuracy.

In some embodiments, generating the predicted leak measure(s) includes generating one or more measures for one or more alternative therapy devices. That is, the leak prediction system may generate a set of device or apparatus-specific measures, such as one predicted leak measure for a full-face mask, one predicted leak measure for a nasal-only mask, one predicted leak measure for a nasal mask with use a chin strap, and the like. In other embodiments, as discussed, above, the leak prediction system generates a mask-agnostic prediction based on the patient's attributes, without consideration for the specific therapy apparatuses used.

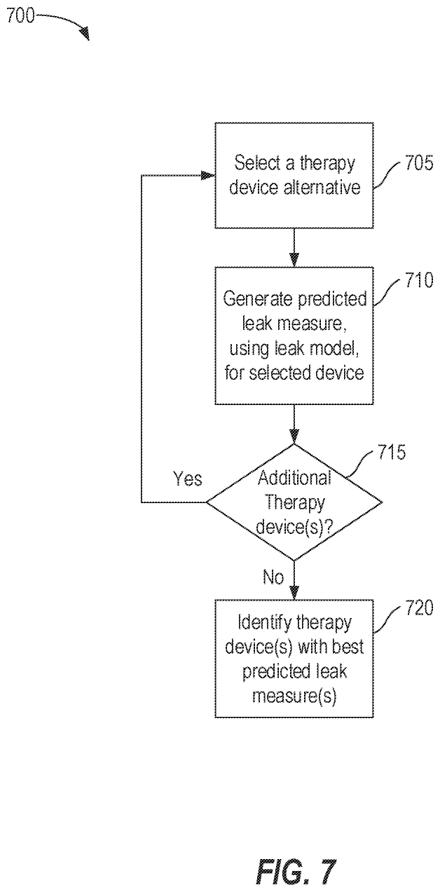

At block 420, the leak prediction system identifies an optimal therapy device based on the predicted leak measure(s). For example, if the leak prediction system generates apparatus-specific predictions, the leak prediction system may identify the therapy apparatus (or combination of apparatuses) having the best leak measures (e.g., the lowest probability of mouth leak, the lowest predicted volume of mouth leak, and the like). In some embodiments, if the predicted leak measure is device-agnostic, the leak prediction system may compare it against defined criteria to identify the optimal device. For example, if the measure includes a probability of mouth leak and/or volume of mouth leak, the leak prediction system may determine whether these values meet or exceed defined criteria. If so, the leak prediction system may identify a full face mask and/or chin strap as the optimal setup for the patient. One example technique for generating the predicted mouth leak measure(s) and/or identifying the optimal device is discussed in more detail below with reference to FIG. 7.

At block 425, the leak prediction system facilitates provisioning of the optimal therapy device for the patient. This may include, for example, outputting a suggestion or indication of the identified optimal device, ordering or otherwise requesting the therapy device, and the like. As discussed above, the leak prediction system can thereby enable improved therapy results and reduced adverse effects in patients.

### Example Method for Extracting Features from Patient Facial Data

FIG. 5 is a flow diagram depicting an example method 500 for extracting features from patient facial data. In some embodiments, the method 500 is performed by a leak prediction system, such as leak prediction system 115 of FIG. 1, and/or leak prediction system 215 of FIG. 2. In at least one embodiment, the method 500 provides additional detail for block 320 of FIG. 3 and/or block 410 of FIG. 4.

At block 505, the leak prediction system determines one or more facial attributes of the patient. As used herein, facial attributes can generally include data relating to the anatomy, structure, shape, and/or geometry of the patient's face, head, and/or neck. For example, the facial attributes may include data such as the thickness and/or size of the patient's upper and/or lower lips, the length, depth, and/or angle of the mentolabial sulcus of the patient, the volume of the upper and/or lower pars marginalis muscles of the patient, the mechanics of the mandible and connected tissues, data relating to the resistance to air flow through the nasal passage of the patient, and the like. As discussed above, the leak prediction system may determine some or all of these attributes explicitly (e.g., where the patient data explicitly indicates them) and/or may determine some or all using inference or other techniques (e.g., by evaluating images or scans of the patient's face).

At block 510, the leak prediction system determines one or more physical attributes of the patient. As used herein, physical attributes can generally include data relating to physical characteristics of the patient (including demographics and/or biographical data), such as their age, height, weight, BMI, mass distributions, and the like. As discussed above, the leak prediction system may determine some or all of these attributes explicitly (e.g., where the patient data explicitly indicates them) and/or may determine some or all using inference or other techniques (e.g., by evaluating images or scans of the patient's face).

At block 515, the leak prediction system determines one or more attributes of therapy data for the patient. As used herein, therapy data can generally include data relating to the therapy preferences and/or medical history of the patient, such as their preferred sleeping position, whether they have previously used (or currently use) a PAP device, whether they have previously experienced (or are currently experiencing) therapy issues such as mouth leak, and the like. As discussed above, the leak prediction system may determine some or all of these attributes explicitly (e.g., where the patient data explicitly indicates them) and/or may determine some or all using inference or other techniques (e.g., by evaluating images or scans of the patient's face).

Although the illustrated example depicts general groups of patient attributes for conceptual clarity, in embodiments, the leak prediction system may use any number and variety of patient attributes with the leak model. As discussed above, the specific features and attributes used may vary depending on the particular implementation of the leak model.

### Example Method for Extracting Features from Patient Informational Data

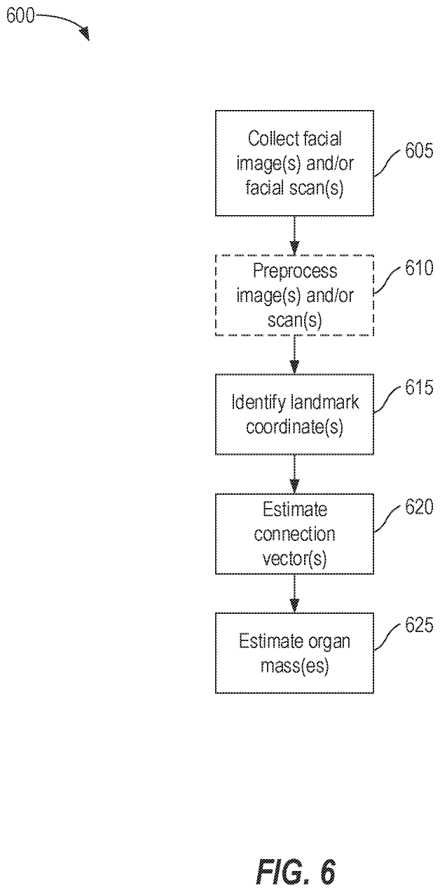

FIG. 6 is a flow diagram depicting an example method 600 for extracting features from patient informational data. In some embodiments, the method 600 is performed by a leak prediction system, such as leak prediction system 115 of FIG. 1, and/or leak prediction system 215 of FIG. 2. In at least one embodiment, the method 600 provides additional detail for block 320 of FIG. 3, block 410 of FIG. 4, and/or block 505 of FIG. 5.

At block 605, the leak prediction system collects, receives, or otherwise accesses, one or more facial images and/or scans of the patient. As used herein, a facial image generally corresponds to a two-dimensional image or sequence of images (e.g., a video), such as captured using an imaging device (e.g., a camera), depicting all or a portion of the patient's face, head, and/or neck. As used herein, a facial scan generally corresponds to a two-dimensional or three-dimensional scan or sequence of scans, such as captured using a scanning device and/or generated using images captured by an imaging device, depicting all or a portion of the patient's face, head, and/or neck.

In at least one embodiment, the collected images and/or scans include a frontal image (e.g., depicting the patient from the front, where the patient is looking towards the imaging sensor) and one or more profile images (e.g., depicting the patient from the side, where the patient is looking towards the left or right of the imaging sensor).

At block 610, the leak prediction system optionally preprocesses the image(s) and/or scan(s). For example, as discussed above, the leak prediction system may perform segmentation based on the nose of the patient (e.g., to crop the image to the patient's nose, thereby excluding irrelevant or non-useful data). As additional examples, the optional preprocessing may include operations such as color-balancing, conversion to grayscale, and the like.

At block 615, the leak prediction system identifies one or more landmark coordinates in the image(s) and/or scan(s). For example, as discussed above, the leak prediction system may use a trained machine learning model and/or a predefined model of a human face to identify or estimate the spatial coordinates (e.g., in two or three-dimensional space) of a defined set of landmarks, such as the fulcrum of the patient's mandible, the connection point(s) of one or more muscles or other organs (such as fat or other tissues) to the user's mandible and/or skull, and the like. For example, the leak prediction system may process the images using a face mesh model including defined landmarks are pre-programmed points. These points can then be used to measure the distances (e.g., the geodesic and/or Euclidean distance) and/or angles between facial features.

In some aspects, once the face mesh and/or landmarks are identified or generated, the leak prediction system may further define a shape model (e.g., a function or combination of functions that define an enclosing surface), for one or more organs, and fit the shape model surface to a shape model of one or more adjacent organs, thereby computing or estimating the volume enclosed (e.g., using finite element methods) to evaluate the facial data.

At block 620, the leak prediction system estimates connection vector(s) based on the identified landmark coordinates. For example, based on the point(s) where a given muscle connects, the leak prediction system may determine a vector in two or three-dimensional space where force from the muscle is exerted along the vector. Similarly, based on the point(s) where other organs (e.g., fatty tissue) are connected and one or more known or alternative sleeping positions, the leak prediction system may determine or estimate the vectors along which gravitational forces will be applied (e.g., to the mandible).

At block 625, the leak prediction system can estimate the organ mass(es) of one or more organs based on the landmark coordinates, facial images/scans, and/or other patient data. For example, as discussed above, the leak prediction system may determine the average or median mass of a given muscle or other organ, based on the patient's age, weight, height, sex, and the like. Similarly, using image recognition, the leak prediction system may evaluate the images to infer these masses.

As discussed above, based on the connection vectors and organ masses, the leak prediction system can thereby predict or infer the amount of force that will pull the mandible open (e.g., due to gravitational force). Similarly, by evaluating the muscle mass and connection points, the leak prediction system can predict or infer the amount of force that will hold the mandible closed while sleeping. By comparing these forces, the leak prediction system can predict whether the patient's mouth will remain closed, and thereby predict whether mouth leak will occur (and, in some cases, the probably amount of mouth leak).

### Example Method for Generating Predicted Leak Measures for Therapy Devices Using Leak Models

FIG. 7 is a flow diagram depicting an example method 700 for generating predicted leak measures for therapy devices using leak models. In some embodiments, the method 700 is performed by a leak prediction system, such as leak prediction system 115 of FIG. 1, and/or leak prediction system 215 of FIG. 2. In at least one embodiment, the method 700 provides additional detail for blocks 415 and 420 of FIG. 4.

At block 705, the leak prediction system selects one of a set of alternative therapy devices. As discussed above, this may generally include selecting a combination of therapy components from a defined set of alternatives, such as selecting a mask type (e.g., full-face, nasal only, pillow, and the like), selecting additional apparatuses (e.g., whether a chin strap is used, whether a humidifier is used, and the like), whether additional therapy aid is used (e.g., coaching or other advice from an expert), and the like. Generally, the leak prediction system can select the alternative using any suitable technique, including randomly or pseudo-randomly, as the leak prediction system will evaluate all alternatives using the method 700.

At block 710, the leak prediction system generates a predicted leak measure, using a leak model, based on the selected device alternative and the patient data that is currently being processed. For example, as discussed above, the leak prediction system may input patient data and the selected therapy device to the model in order to generate an apparatus-specific prediction as to whether the therapy will result in mouth leak, the predicted volume of such mouth leak, and the like.

At block 715, the leak prediction system determines whether there is at least one alternative therapy device (or combination thereof) that has not-yet been evaluated. If so, the method 700 returns to block 705. If not, the method 700 continues to block 720, where the leak prediction system identifies the therapy device(s) that are best suited for the patient, based on the predicted mouth leak measures. For example, as discussed above, the leak prediction system may identify the therapy device(s) having the lowest probability of mouth leak, the lowest (zero or non-zero) predicted volume of mouth leak, and the like.

In this way, the leak prediction system can enable selection of the most optimal therapy device(s) for the specific patient, thereby significantly improving patient results and outcomes.

In some examples, mouth leak during PAP therapy id described as an example therapy issue that can be predicted using embodiments of the present disclosure. In some embodiments, the disclosed architectures, techniques, and operations can be used for a variety of issues. For example, aspects of the present disclosure may be used to estimate or predict the appropriateness of any therapy options, such as the CPAP apparatus to be used, alternative treatments that might work for the patient (e.g., mandibular advancement device, positional therapy, nerve stimulation, specific pharmaceutical medications, psychological or cognitive behavioral therapies, or even no therapy at all), and the like.

### Example Method for Training Leak Models

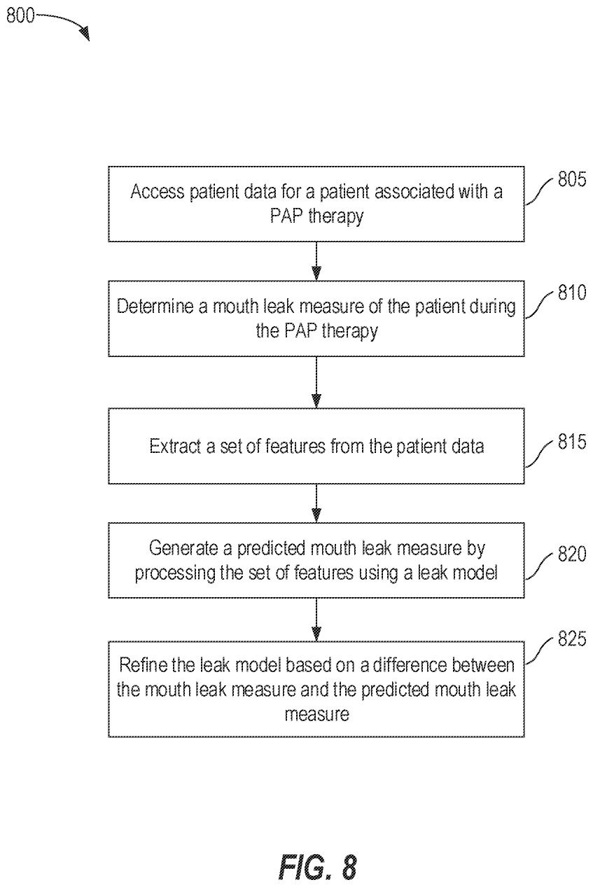

FIG. 8 is a flow diagram depicting an example method 800 for training leak models. In some embodiments, the method 800 is performed by a leak prediction system, such as leak prediction system 215 of FIG. 2, and/or leak prediction system 115 of FIG. 1.

At block 805, patient data (e.g., patient data 205 of FIG. 2) for a patient associated with a PAP therapy is accessed.

At block 810, a mouth leak measure (e.g., leak data 210 of FIG. 2) of the patient during the PAP therapy is determined.

At block 815, a set of features is extracted from the patient data.

At block 820, a predicted mouth leak measure is generated by processing the set of features using a leak model (e.g., leak model 230 of FIG. 2).

At block 825, the leak model is refined based on a difference between the mouth leak measure and the predicted mouth leak measure.

### Example Method for Provisioning of Therapy Devices Using Leak Models

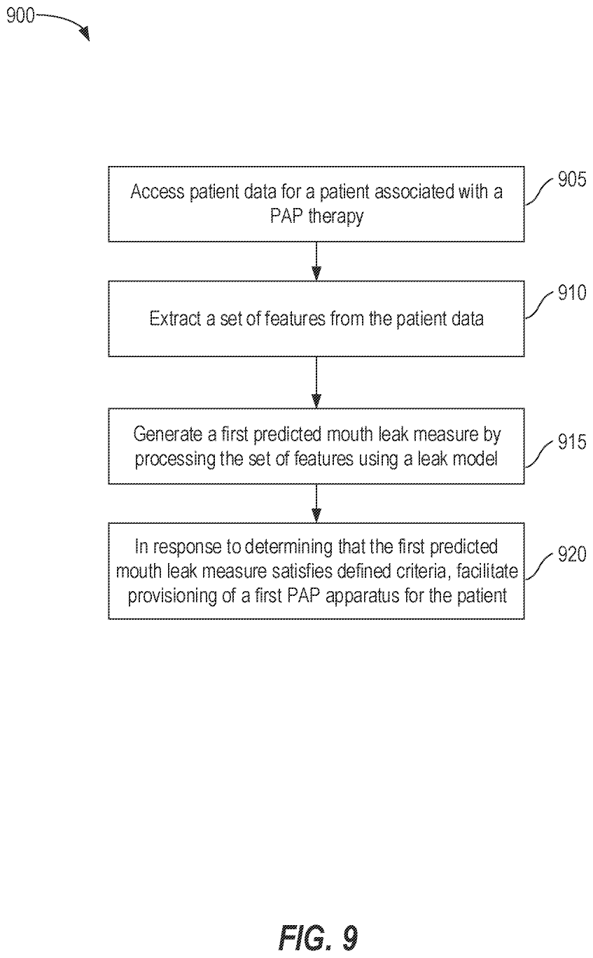

FIG. 9 is a flow diagram depicting an example method 900 for facilitating provisioning of therapy devices using leak models. In some embodiments, the method 900 is performed by a leak prediction system, such as leak prediction system 115 of FIG. 1, and/or leak prediction system 215 of FIG. 2.

At block 905, patient data (e.g., patient data 105 of FIG. 1) for a patient associated with a PAP therapy is accessed.

At block 910, a set of features is extracted from the patient data.

At block 915, a predicted mouth leak measure is generated by processing the set of features using a leak model (e.g., leak model 130 of FIG. 1).

At block 920, in response to determining that the first predicted mouth leak measure satisfies defined criteria, provisioning of a first PAP apparatus (e.g., therapy device 135 of FIG. 1) for the patient is facilitated.

### Example Computing Device for Leak Prediction

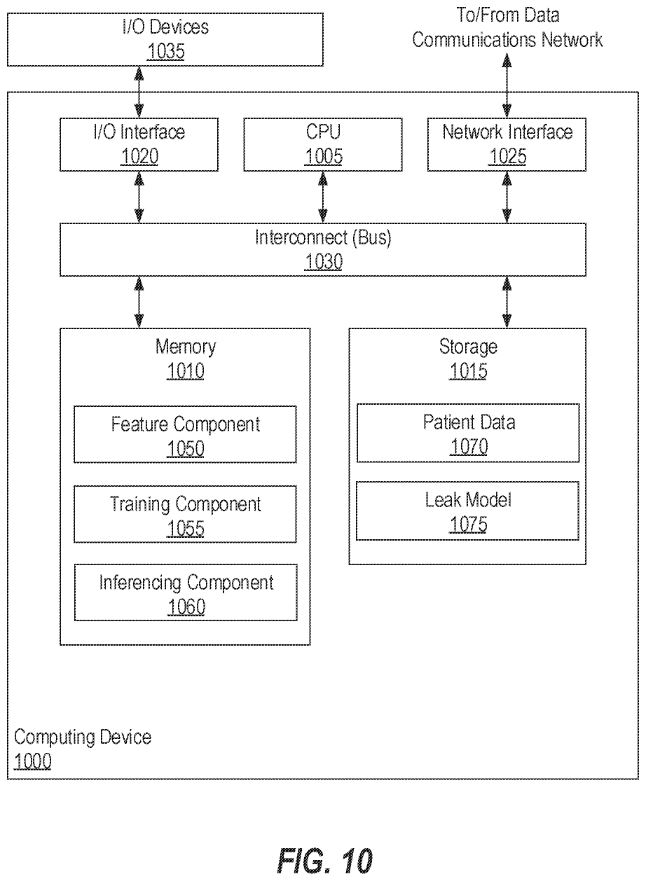

FIG. 10 depicts an example computing device 1000 configured to perform various aspects of the present disclosure. Although depicted as a physical device, in embodiments, the computing device 1000 may be implemented using virtual device(s), and/or across a number of devices (e.g., in a cloud environment). In one embodiment, the computing device 1000 corresponds to a leak prediction system such as the leak prediction system 115 of FIG. 1 and/or the leak prediction system 215 of FIG. 2.

As illustrated, the computing device 1000 includes a CPU 1005, memory 1010, storage 1015, a network interface 1025, and one or more I/O interfaces 1020. In the illustrated embodiment, the CPU 1005 retrieves and executes programming instructions stored in memory 1010, as well as stores and retrieves application data residing in storage 1015. The CPU 1005 is generally representative of a single CPU and/or GPU, multiple CPUs and/or GPUs, a single CPU and/or GPU having multiple processing cores, and the like. The memory 1010 is generally included to be representative of a random access memory. Storage 1015 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1035 (such as keyboards, monitors, etc.) are connected via the I/O interface(s) 1020. Further, via the network interface 1025, the computing device 1000 can be communicatively coupled with one or more other devices and components (e.g., via a network, which may include the Internet, local network(s), and the like). As illustrated, the CPU 1005, memory 1010, storage 1015, network interface(s) 1025, and I/O interface(s) 1020 are communicatively coupled by one or more buses 1030.

In the illustrated embodiment, the memory 1010 includes a feature component 1050, a training component 1055, and an inferencing component 1060, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1010, in embodiments, the operations of the depicted components (and others not illustrated) may be implemented using hardware, software, or a combination of hardware and software.

In one embodiment, the feature component 1050 (which may correspond to the feature extractor 120 of FIG. 1) may be used to perform preprocessing and/or extract features from input data, as discussed above. For example, the feature component 1050 may be used to segment image data, extract or determine facial anatomy, and the like. The training component 1055 (which may correspond to the training component 225 of FIG. 2) may generally be used to generate or train machine learning models (e.g., leak models 130 of FIG. 1 and/or leak models 230 of FIG. 2), as discussed above. For example, the training component 1055 may refine or update model parameters based on historical patient data in order to generate improved models that can better-predict patient mouth leak. The inferencing component 1060 (which may correspond to the inferencing component 125 of FIG. 1) may generally be used to generate predicted mouth leak using trained models, and/or to facilitate provisioning of therapy devices, as discussed above. For example, the inferencing component 1060 may process patient features using leak models to predict mouth leak, and identify optimal combinations of therapy apparatuses.

In the illustrated example, the storage 1015 includes patient data 1070, one or more leak models 1075. In one embodiment, the patient data 1070 (which may correspond to the patient data 105 of FIG. 1, the patient data 205 of FIG. 2, and/or the leak data 210 of FIG. 2) may include attributes or characteristics of patients, as discussed above. For example, the patient data 1070 can generally include attributes of patient(s) associated with PAP therapy (e.g., those engaged in it or who are considering engaging in it), and may generally include information that may be predictive of mouth leak. The leak model 1075 (which may correspond to the leak model 130 of FIG. 1 and/or the leak model 230 of FIG. 2) generally corresponds to a computational model (e.g., a machine learning model) that generates a predicted leak measurement based on input data. Although depicted as residing in storage 1015, the patient data 1070 and leak model 1075 may be stored in any suitable location, including memory 1010.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., leak prediction system 115 of FIG. 1 and/or leak prediction system 215 of FIG. 2) or related data available in the cloud. For example, the leak prediction system could execute on a computing system in the cloud and automatically generate mouth leak predictions based on patient data. In such a case, the interface system could use patient-specific information to predict whether they will experience mouth leak, and return indications of the predicted mouth leaks and/or suggested devices or techniques to mitigate or prevent such leaks. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### Example Clauses

Implementation examples are described in the following numbered clauses:

Clause 1: A method, comprising: accessing patient data for a patient associated with a positive airway pressure (PAP) therapy; determining a mouth leak measure of the patient during the PAP therapy; extracting a set of features from the patient data; generating a predicted mouth leak measure by processing the set of features using a leak model; and refining the leak model based on a difference between the mouth leak measure and the predicted mouth leak measure.

Clause 2: The method of Clause 1, wherein extracting the set of features comprises determining facial information comprising: a size and a shape of a nose of the patient; and a predicted measure of nasal resistance to airflow for the patient, wherein the predicted measure of nasal resistance is determined using acoustic impedance measurement.

Clause 3: The method of any one of Clauses 1-2, wherein the patient data comprises at least one of an image of the patient or a three-dimensional scan of the patient.

Clause 4: The method of any one of Clauses 1-3, wherein the leak model comprises a convolutional neural network machine learning model.

Clause 5: The method of any one of Clauses 1-4, wherein extracting the set of features comprises estimating a gravitational force on a mandible of the patient, comprising: estimating a spatial coordinate of a fulcrum of the mandible; estimating spatial coordinates of one or more connection points of one or more organs of the patient to the mandible; determining one or more attributes of the patient based on the patient data; and estimating a mass of the one or more organs based on fitting the one or more attributes to an organ model.

Clause 6: A method, comprising: accessing patient data for a patient associated with a positive airway pressure (PAP) therapy; extracting a set of features from the patient data; generating a first predicted mouth leak measure by processing the set of features using a leak model; and in response to determining that the first predicted mouth leak measure satisfies defined criteria, facilitating provisioning of a first PAP apparatus for the patient.

Clause 7: The method of Clause 6, wherein: the patient is engaged in the PAP therapy using a second PAP apparatus; and the first predicted mouth leak measure is further generated by processing an indication of the second PAP apparatus using the leak model.

Clause 8: The method of any one of Clauses 6-7, wherein facilitating provisioning of the first PAP apparatus comprises: determining, based on the plurality of predicted mouth leak measures, that the first PAP apparatus is least likely to cause mouth leak.

Clause 9: The method of any one of Clauses 6-8, wherein extracting the set of features comprises determining facial information indicating a size and a shape of a nose of the patient.

Clause 10: The method of any one of Clauses 6-9, wherein the facial information further comprises a predicted measure of nasal resistance to airflow for the patient, wherein the predicted measure of nasal resistance is determined using acoustic impedance measurement.

Clause 11: The method of any one of Clauses 6-10, wherein the patient data comprises at least one of an image of the patient or a three-dimensional scan of the patient.

Clause 12: The method of any one of Clauses 6-11, wherein the leak model comprises a convolutional neural network machine learning model.

Clause 13: The method of any one of Clauses 6-12, wherein extracting the set of features comprises segmenting the patient data based on a nose of the patient.

Clause 14: The method of any one of Clauses 6-13, wherein extracting the set of features comprises estimating a gravitational force on a mandible of the patient.

Clause 15: The method of any one of Clauses 6-14, wherein estimating the gravitational force comprises: estimating a spatial coordinate of a fulcrum of the mandible; and estimating spatial coordinates of one or more connection points of one or more organs of the patient to the mandible.

Clause 16: The method of any one of Clauses 6-15, wherein estimating the gravitational force further comprises: determining one or more attributes of the patient based on the patient data; and estimating a mass of the one or more organs based on fitting the one or more attributes to an organ model.

Clause 17: The method of any one of Clauses 6-16, wherein estimating the gravitational force further comprises determining a sleeping position of the patient.

Clause 18: A system, comprising: a memory comprising computer-executable instructions; and one or more processors configured to execute the computer-executable instructions and cause the processing system to perform a method in accordance with any one of Clauses 1-17.

Clause 19: A system, comprising means for performing a method in accordance with any one of Clauses 1-17.

Clause 20: A non-transitory computer-readable medium comprising computer-executable instructions that, when executed by one or more processors of a processing system, cause the processing system to perform a method in accordance with any one of Clauses 1-17.

Clause 21: A computer program product embodied on a computer-readable storage medium comprising code for performing a method in accordance with any one of Clauses 1-17.

What is claimed is:

1. A method, comprising:
accessing patient data for a patient associated with a positive airway pressure (PAP) therapy;
determining a mouth leak measure of the patient during the PAP therapy;
extracting a set of features from the patient data;
generating a predicted mouth leak measure by processing the set of features using a leak model; and
refining the leak model based on a difference between the mouth leak measure and the predicted mouth leak measure.

2. The method of claim 1, wherein extracting the set of features comprises determining facial information comprising:
a size and a shape of a nose of the patient; and
a predicted measure of nasal resistance to airflow for the patient, wherein the predicted measure of nasal resistance is determined using acoustic impedance measurement.

3. The method of claim 1, wherein the patient data comprises at least one of an image of the patient or a three-dimensional scan of the patient.

4. The method of claim 3, wherein the leak model comprises a convolutional neural network machine learning model.

5. The method of claim 1, wherein extracting the set of features comprises estimating a gravitational force on a mandible of the patient, comprising:
estimating a spatial coordinate of a fulcrum of the mandible;
estimating spatial coordinates of one or more connection points of one or more organs of the patient to the mandible;
determining one or more attributes of the patient based on the patient data; and
estimating a mass of the one or more organs based on fitting the one or more attributes to an organ model.

6. A method, comprising:
accessing patient data for a patient associated with a positive airway pressure (PAP) therapy;
extracting a set of features from the patient data;
generating a first predicted mouth leak measure by processing the set of features using a leak model; and
in response to determining that the first predicted mouth leak measure satisfies defined criteria, facilitating provisioning of a first PAP apparatus for the patient.

7. The method of claim 6, wherein:
the patient is engaged in the PAP therapy using a second PAP apparatus; and
the first predicted mouth leak measure is further generated by processing an indication of the second PAP apparatus using the leak model.

8. The method of claim 7, wherein facilitating provisioning of the first PAP apparatus comprises:
generating a plurality of predicted mouth leak measures by, for each respective PAP apparatus of a plurality of PAP apparatuses, processing a respective indication of the respective PAP apparatus and the set of features using the leak model; and
determining, based on the plurality of predicted mouth leak measures, that the first PAP apparatus is least likely to cause mouth leak.

9. The method of claim 6, wherein extracting the set of features comprises determining facial information indicating a size and a shape of a nose of the patient.

10. The method of claim 9, wherein the facial information further comprises a predicted measure of nasal resistance to airflow for the patient, wherein the predicted measure of nasal resistance is determined using acoustic impedance measurement.

11. The method of claim 6, wherein the patient data comprises at least one of an image of the patient or a three-dimensional scan of the patient.

12. The method of claim 11, wherein the leak model comprises a convolutional neural network machine learning model.

13. The method of claim 12, wherein extracting the set of features comprises segmenting the patient data based on a nose of the patient.

14. The method of claim 6, wherein extracting the set of features comprises estimating a gravitational force on a mandible of the patient.

15. The method of claim 14, wherein estimating the gravitational force comprises:
estimating a spatial coordinate of a fulcrum of the mandible; and
estimating spatial coordinates of one or more connection points of one or more organs of the patient to the mandible.

16. The method of claim 15, wherein estimating the gravitational force further comprises:
determining one or more attributes of the patient based on the patient data; and
estimating a mass of the one or more organs based on fitting the one or more attributes to an organ model.

17. The method of claim 15, wherein estimating the gravitational force further comprises determining a sleeping position of the patient.

18. A non-transitory computer-readable medium comprising computer-executable instructions that, when executed by one or more processors of a processing system, cause the processing system to perform an operation comprising:
accessing patient data for a patient associated with a positive airway pressure (PAP) therapy;
extracting a set of features from the patient data;
generating a first predicted mouth leak measure by processing the set of features using a leak model; and
in response to determining that the first predicted mouth leak measure satisfies defined criteria, facilitating provisioning of a first PAP apparatus for the patient.

19. The non-transitory computer-readable medium of claim 18, wherein:
the patient data comprises at least one of an image of the patient or a three-dimensional scan of the patient, and
the leak model comprises a convolutional neural network machine learning model.

20. The non-transitory computer-readable medium of claim 18, wherein extracting the set of features comprises estimating a gravitational force on a mandible of the patient, comprising:
estimating a spatial coordinate of a fulcrum of the mandible;
estimating spatial coordinates of one or more connection points of one or more organs of the patient to the mandible;
determining one or more attributes of the patient based on the patient data; and
estimating a mass of the one or more organs based on fitting the one or more attributes to an organ model.
